(19) United States
(12) Patent Application Publication
(10) Pub. No.: US 2024/0374570 A1
(43) Pub. Date: Nov. 14, 2024

(54) DRIVING AXON REGENERATION BY NRF2 OVEREXPRESSION AND EDARAVONE APPLICATION

(71) Applicant: The Hong Kong University of Science and Technology, Hong Kong (CN)

(72) Inventors: Kai LIU, Hong Kong (CN); Chao YANG, Hong Kong (CN); Leung Ting CHAN, Hong Kong (CN); Mengxian CHEN, Hong Kong (CN)

(21) Appl. No.: 18/657,915

(22) Filed: May 8, 2024

Related U.S. Application Data
(60) Provisional application No. 63/500,622, filed on May 8, 2023.

(51) Int. Cl. A61K 31/4152; A61K 9/00; A61K 48/00; A61P 25/28

(52) U.S. Cl. CPC A61K 31/4152; A61K 9/0048; A61K 48/005; A61K 48/0075; A61P 25/28

(57) ABSTRACT

The subject invention pertains to a method for promoting axon regeneration in a subject, including those with central nervous system (CNS) injury, by activation of Nrf2 induced by edaravone.

### CROSS-REFERENCE TO RELATED APPLICATION

The subject application claims the benefit of U.S. Provisional Application Ser. No. 63/500,622, filed May 8, 2023, which is incorporated herein by reference in its entirety.

### BACKGROUND OF THE INVENTION

Axons do not spontaneously regenerate after injury to the adult mammalian central nervous system. This phenomenon leads to persistent dysfunction of the nervous system after injury and limits the development of repair treatments for the central nervous system (CNS) injury.

Extrinsic and intrinsic factors work together to impede axon regeneration after injury to the CNS. CNS injury triggers reactive astrocytes proliferation and chondroitin sulfate proteoglycans (CSPGs) secretion at lesion site and contribute to the glia scar formation [1, 2]. Myelin associated molecules (such as Nogo and MAG) are another major component that contributes to form an inhibitory environment for axon regeneration [3, 4].

The inhibitory effect of these extrinsic inhibitors has been investigated by pharmacological or genetic-mediated elimination. Previous studies suggest that multiple neuronal intrinsic pathways could be the targets for developing therapeutic strategies [5]. However, clinically applicable drugs targeting these pathways only show minor effect on axon regeneration, which precludes their application for clinical treatment [6-9].

Several intrinsic factors associated with axon regeneration have been discovered, e.g., PTEN [10], Socs3 [11], B-RAF [12], c-Myc [13] and Lin28 [14]. However, there is still a lack of highly potent inhibitors or activators targeting these intrinsic factors. In addition, most of these intrinsic factors are tumor suppressors or oncogenes, and a deletion or activation of these genes may disrupt intracellular homeostasis, impair neuronal function [15] or induce tumorigenesis [16-18]. These side effects strongly hinder the clinical application of the discovered intrinsic factors. Currently existing treatments are still struggling to meet the requirements for clinical trials. As a result, there are no clinically applicable drugs for functional restoration therapy after CNS injuries. Therefore, there is a need for a safe and effective functional restoration therapy after CNS injuries.

### BRIEF SUMMARY OF THE INVENTION

The present invention provides a method for promoting axon regeneration in a subject with central nervous system (CNS) injury by nuclear factor erythroid 2-related factor 2 (Nrf2) activation.

In preferred embodiments, Nrf2 activation is induced by edaravone (5-methyl-2-phenyl-2,4-dihydro-3H-pyrazol-3-one).

In some embodiments, a therapeutically effective amount of edaravone is administered to a subject.

In further embodiments, the present invention is a method of treating a subject with central nervous system (CNS) injury, by Nrf2 activation, comprising administering to the subject in need thereof a therapeutically effective amount of edaravone, where Nrf2 activation is induced by edaravone.

In preferred embodiments, the administration of edaravone to a subject upregulates Nrf2, which binds to antioxidant response element (AR) sites, and the upregulation of Nrf2 reduces the production of reactive-oxygen species (ROS).

In more preferred embodiments, overexpression of Nrf2 significantly promotes axon regeneration after optic nerve injury.

In some embodiments, edaravone is administered to a subject with CNS injury, locally by intravitreal, intracranial, intradiscal, or systemically, by intravenous or intraperitoneal injection, where edaravone administration enhances neuron survival and promotes axon growth in the subject.

In some embodiments, the subject is a mammal, preferably the mammal is a primate, and more preferably the primate is a human.

In some embodiments, the CNS injury can be is spinal cord injury, traumatic brain injury, optic neuropathy, stroke, or glaucoma.

In preferred embodiments, the edaravone is administered to the subject at a dose of about 0.1 mg/kg to about 100 mg/kg, and more preferably at a dose of about 5 mg/kg to about 80 mg/kg.

In more preferred embodiments, the edaravone is administered to the subject at a dose of 60 mg/day via 60 minute IV infusion.

In preferred embodiments, wherein edaravone is administered in a composition at a concentration of about 0.1 μg/mL to about 10 mg/mL.

In other embodiments, Nrf2 activation can be induced by AAV-mediated Nrf2 overexpression or Nrf2 activators.

### BRIEF DESCRIPTION OF THE DRAWINGS

FIGS. 1A-1B Nrf2 overexpression promotes optic nerve regeneration. (FIG. 1A) Sections of optic nerves from WT mice at 2 weeks post injury. The vitreous body was injected with either AAV2-GFP control or AAV2-Nrf2. Axons were labeled by CTB-FITC. Scale bar: 200 μm. (FIG. 1B) Quantification of regenerating axons at indicated distances from the lesion site in FIG. 1A. **p≤0.01. ANOVA followed by Bonferroni's test, n=4-5 mice.

FIGS. 2A-2B Nrf2 overexpression enhances RGC survival after optic nerve injury. (FIG. 2A) Whole-mount retinas from WT mice at 2 weeks post injury with Tuj1 staining. The vitreous body was injected with either AAV2-GFP control or AAV2-Nrf2. Scale bar: 50 μm. (FIG. 2B) Quantification of RGC survival in FIG. 2A. **p≤0.01. Student's t-test, n=4-5 mice.

FIGS. 3A-3B Edaravone promotes axon elongation in DRG replating culture. (FIG. 3A) Representative image of replated DRG neurons treated by the DMSO vehicle control and indicated concentration of edaravone. Scale bar: 400 μm. (FIG. 3B) Quantification of the longest neuron length of the tested concentrations of edaravone. Dots represent the measurement of each neuron.

FIGS. 4A-4B Edaravone daily treatment increases RGC survival after optic nerve injury. (FIG. 4A) Whole-mount retinas from WT mice at 2 weeks post injury with Tuj1 staining. The mice received vehicle or varying dosages of edaravone IP injection. (FIG. 4B) Quantification of RGC survival in FIG. 4A. **p≤0.01.

FIG. 5 Summary of the factors for converting doses between animals and humans.

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” are used, these compositions contain the stated amount of the ingredient with a variation (error range) of 0-10% around the value (X±10%). In other contexts, the term “about” is providing a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, “subject”, “host” or “organism” refers to any member of the phylum Chordata, more preferably any member of the subphylum vertebrata, or most preferably, any member of the class Mammalia, including, without limitation, humans and other primates, including non-human primates such as rhesus macaques, chimpanzees and other monkey and ape species; livestock, such as cattle, sheep, pigs, goats and horses; domestic mammals, such as dogs and cats; laboratory animals, including rabbits, mice, rats and guinea pigs. The term does not denote a particular age or gender. Thus, adult, young, and new-born individuals are intended to be covered as well as male and female subjects. In some embodiments, a host tissue is derived from a subject. In some embodiments, the subject is a non-human subject.

As used herein, the terms “therapeutically-effective amount,” “therapeutically-effective dose,” “effective amount,” and “effective dose” are used to refer to an amount or dose of a compound or composition that, when administered to a subject, is capable of treating, preventing, or improving a condition, disease, or disorder in a subject. In other words, when administered to a subject, the amount is “therapeutically effective.” The actual amount will vary depending on several factors including, but not limited to, the particular condition, disease, or disorder being treated, prevented, or improved; the severity of the condition; the weight, height, age, and health of the patient; and the route of administration.

As used herein, the term “treatment” refers to eradicating; reducing; ameliorating; abatement; remission; diminishing of symptoms or delaying the onset of symptoms; slowing in the rate of degeneration or decline; making the final point of degeneration less debilitating; and/or improving a subject's physical or mental well-being or reversing a sign or symptom of a health condition, disease or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

As used herein, “preventing” a health condition, disease, or disorder refers to avoiding, delaying, forestalling, or minimizing the onset of a particular sign or symptom of the condition, disease, or disorder. Prevention can, but is not required, to be absolute or complete; meaning, the sign or symptom may still develop at a later time. Prevention can include reducing the severity of the onset of such a condition, disease, or disorder, and/or inhibiting the progression of the condition, disease, or disorder to a more severe condition, disease, or disorder.

In some embodiments of the invention, the method comprises administration of multiple doses of the compounds of the subject invention. The method may comprise administration of 1, 2, 3, 4, 5, 6, 7, 8, 9, 10, 15, 20, 25, 30, 35, 40, 50, 60, 70, 80, 90, 100 or more therapeutically effective doses of a composition comprising the compounds of the subject invention as described herein. In some embodiments, doses are administered over the course of 1 day, 2 days, 3 days, 4 days, 5 days, 6 days, 7 days, 10 days, 14 days, 21 days, 30 days, 2 months, 3 months, 6 months, 9 months, 1 year, 1.5 years, 2 years, 2.5 years, 5 years, or more than 10 years. The frequency and duration of administration of multiple doses of the compositions is such as prevent or treat endothelial dysfunction. Moreover, treatment of a subject with a therapeutically effective amount of the compounds of the invention can include a single treatment or can include a series of treatments. It will also be appreciated that the effective dosage of a compound used for treatment may increase or decrease over the course of a particular treatment. Changes in dosage may result and become apparent from the results of testing for endothelial dysfunction, such as, for example, magnetic resonance imaging or positron emission tomography. In some embodiments of the invention, the method comprises administration of the compounds at several times per day, including but not limiting to 2 times per day, 3 times per day, and 4 times per day.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, a “pharmaceutical” refers to a compound manufactured for use as a medicinal and/or therapeutic drug.

Any compositions or methods provided herein can be combined with one or more of any of the other compositions and methods provided herein.

Other features and advantages of the invention will be apparent from the following description of the preferred embodiments thereof, and from the claims.

All references cited herein are hereby incorporated by reference in their entirety.

The present invention relates to a novel method for promoting axon regeneration in a subject with CNS injury by activation of therapeutic target nuclear factor erythroid 2-related factor 2 (Nrf2). Nrf2 overexpression significantly promotes axon regeneration after CNS nerve injury. Edaravone, an FDA-approved antioxidative drug is an Nfr2 activator that is effective in promoting axon growth.

Traumatic injury to the CNS results in permanent dysfunction of the motor and sensory systems, the main reason being the rare axon ability to regenerate resulting in neuronal loss. Axons do not spontaneously regenerate after injury to the adult mammalian CNS, resulting in persistent dysfunction of the nervous system after injury, and limiting the development for repair treatments of the CNS injury. Although previous studies suggest that multiple neuronal intrinsic pathways could be targets for developing therapeutic strategies [5], clinically applicable drugs targeting these pathways are still lacking.

We identified a novel therapeutic target, nuclear factor erythroid 2-related factor 2 (Nrf2), which significantly promoted axon regeneration after optic nerve injury when overexpressed. In addition, Nrf2 overexpression significantly enhanced retinal ganglion cells (RGCs) survival after injury. Furthermore, screening of Nrf2 activator identified an FDA-approved antioxidative drug, edaravone, which exhibited axon growth-promoting effect and neuronal protection effects on nerve injuries.

Neurons undergo a variety of cellular and signaling events after injury, such as endoplasmic reticulum (ER) stress [19], mitochondrial dysfunction [20], elevation of reactive oxygen species (ROS) [21], alternation of anabolic metabolism pathways and catabolic mechanism pathways [1]. ROS-triggered oxidative stress has been observed after traumatic injury [21], and in the neurodegenerative diseases [22]. As products of cellular oxidative metabolism, ROS play vital roles in regulation of cell signaling, cell proliferation, cell differentiation, cell survival, and inflammation [23]. ROS may display protective or deleterious effects depending on the physiological or pathological condition involved. In case of a traumatic injury, excessive production of ROS from mitochondria leads to the alteration of many macromolecules and impairs neuronal survival and functional restoration. However, capturing and scavenging ROS may ameliorate the deterioration process of the disease.

Nrf2 is a transcription factor that binds to antioxidant response element (ARE) sites in the nucleus upon activation and regulates the expression of many genes with roles as antioxidants [24]. Edaravone, an antioxidative drug approved by FDA, is used for the treatment of neurodegenerative disease amyotrophic lateral sclerosis (ALS) [25]. We found that Edaravone treatment reduces the generation of ROS by upregulating antioxidant gene Nrf2 [26, 27]. We discovered that overexpression of Nrf2 significantly promotes axon regeneration and enhances RGCs survival after optic nerve injury. Furthermore, by screening Nrf2 activators, we found that edaravone, an FDA-approved drug, boosted axon growth.

In some embodiments, Nrf2 is effective in significantly promoting axon regeneration and providing neuronal protection after CNS injury.

In preferred embodiments, edaravone promotes axon growth after replating injury.

In more preferred embodiments, edaravone can crosses the blood-brain barrier and target central nervous system neurons.

In some embodiments, edaravone reduces ROS and promotes axon growth, thus providing an alternative approach for clinical trials when compared to genetic overexpression of Nrf2.

In further embodiments, Nrf2 activation can be induced by adeno-associated virus (AAV) mediated overexpression.

In some embodiments, edaravone can be administered in a composition at a concentration of about 0.1 μg/mL to about 10 mg/mL.

In some embodiments, edaravone can be administered at a dose of about 0.1 mg/kg to about 100 mg/kg, about 0.25 mg/kg to about 75 mg/kg, or about 50 mg/kg.

In preferred embodiments, edaravone can be administered at a dose of about about 0.25 mg/kg to about 75 mg/kg.

In preferred embodiments, the edaravone is administered to the subject at a dose of about 0.1 mg/kg to about 100 mg/kg, and more preferably at a dose of about 5 mg/kg to about 80 mg/kg.

In more preferred embodiments, the edaravone is administered to the subject at a dose of 60 mg/day via 60 minute IV infusion.

Edaravone may be added to compositions at concentrations of about 0.0001 to about 5% by weight (wt %), preferably about 0.01 to about 0.5 wt %, and most preferably about 0.1 wt %. In another embodiment, edaravone can be in combination with an acceptable carrier and/or excipient, in that edaravone may be presented at concentrations of about 0.0001 to about 5% (v/v), preferably, about 0.01 to about 0.5% (v/v), more preferably, about 0.05 to about 0.25% (v/v), or, most preferably about 0.1% (v/v).

In some embodiments, edaravone can be administered locally by intravitreal, intracranial, intradiscal, or systemically, by intravenous or intraperitoneal injection.

In some embodiments, edaravone can be administered to the subject by intravitreal injection at a dose of about 0.5 mM to about 10 mM.

In some embodiments, an edaravone composition can be administered locally by intravitreal, intracranial, intradiscal, or systemically, by intravenous or intraperitoneal injection.

The composition of the subject invention can also include additives commonly used in medications.

In certain embodiments, the therapeutically effective amount of the composition of the invention can be administered through intraperitoneal administration or by sustained release systems, such as semipermeable matrices of solid hydrophobic polymers containing the compounds of the invention. Administration may be also by way of other carriers or vehicles such as patches, micelles, liposomes, vesicles, implants (e.g. microimplants), synthetic polymers, microspheres, nanoparticles, and the like. In certain embodiments, the compositions may be administered using a nanoparticle to passage the composition through skin.

In certain embodiments, the compositions of the instant invention may be formulated for parenteral administration e.g., by injection, for example, bolus injection, intravenous administration, intraperitoneal administration, or continuous infusion. In addition, the compositions may be presented in unit dose form in ampoules, pre-filled syringes, and small volume infusion or in multi-dose containers with or without an added preservative. The compositions may be in forms of suspensions, solutions, or emulsions in oily or aqueous vehicles. The composition may further contain formulation agents such as suspending, stabilizing and/or dispersing agents. In further embodiments, the active ingredients of the compositions according to the instant invention may be in powder form, obtained by aseptic isolation of sterile solid or by lyophilization from solution for constitution with a suitable vehicle, e.g., sterile, pyrogen-free water, before use.

Regardless of the route of administration selected, the composition may be formulated into pharmaceutically acceptable dosage form by conventional methods known to those of skill in the art. The composition may be formulated for administration in any convenient way for use in human or veterinary medicine, by analogy with other pharmaceuticals.

The compositions can further comprise one or more pharmaceutically acceptable carriers, and/or excipients, and can be formulated into preparations, for example, semi-solid or liquid forms, such as solutions or injections.

The formulations may conveniently be presented in unit dosage form and may be prepared any methods well known in the art of pharmacy. The amount of compound which can be combined with a carrier material to produce a single dosage form will vary depending upon the subject being treated, the particular mode of administration. The amount of an active ingredient which can be combined with carrier material to produce a single dosage form will usually be the amount of the compound which produces a therapeutic effect. Usually, out of one hundred percent, this amount will range from about 1 wt % to about 99 wt % of active ingredient, preferably from about 5 wt % to about 70 wt %, most preferably from about 10 wt % to about 30 wt %.

The term “pharmaceutically acceptable” as used herein means compatible with the other ingredients of a pharmaceutical composition and not deleterious to the recipient thereof.

Carriers and/or excipients according to the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance or with the composition, carrier or excipient use in the subject compositions may be contemplated.

In preferred embodiments, the administration of at least one dose of the composition is repeated at least daily about 7 weeks or longer. In certain embodiments, the repeated administrations of at least one of dose of the composition occurs for at least about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 52 weeks or longer. In preferred embodiments, the administration of at least one dose of the composition is repeated every about 2 days to about 5 days for about 2 weeks to about 10 weeks.

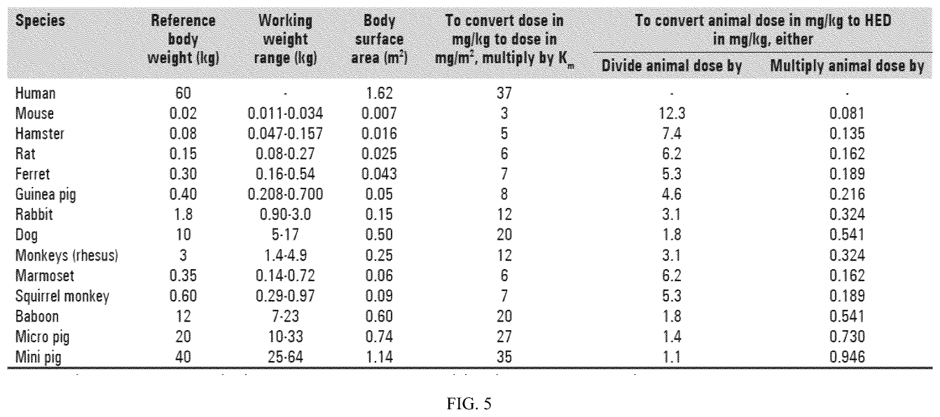

The data obtained from cell culture assays and animal studies may be used in formulating a range of dosage for use in human. For example, effective dosage achieved in one animal species may be extrapolated for use in another animal, including humans, as illustrated in the conversion table of FIG. 5 where human equivalent dose (HED) dosage factors based on body surface area of other species are reported. The dosage of any supplement, or alternatively of any components therein, lies preferably within the range of circulating concentration that include the ED₅₀ with little or no toxicity. The dosage may vary within this range depending upon the dosage form employed and the route of administration utilized. For the compound or combinations of the compound, the therapeutically effective dose may be estimated initially from cell culture assays. A dose may be formulated in animal models to achieve a circulating plasma concentration range that includes the IC₅₀ (i.e., the concentration of the test compound which achieves a half-maximum inhibition of symptoms) as determined in cell culture. Such information may be used to more accurately determine useful doses in humans. Levels of plasma may be measured, for example, by high performance liquid chromatography.

### Materials and Methods

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

The intravitreal injection and optic nerve lesion were performed as following protocols. The mice received a mixture of ketamine (80 mg/kg) and xylazine (10 mg/kg) for anesthesia. The conjunctiva was exposed by clamping the eyelid with an artery clamp. Two microliters of vitreous body were extracted using a Hamilton syringe, and 2 μL of AAV2-hSyn-GFP or AAV2-hSyn-Nrf2 were gently injected into the vitreous body. Four weeks after AAV injection, the optic nerve was exposed and then compressed with Dumont forceps (Dumont #5 and #2; Fine Science Tools) for two seconds to ensure a complete optic nerve injury. Twelve days after the injury, 2 μL of CTB (1 mg/mL, Invitrogen) was injected into the eye to label regenerating axons.

Edaravone IP injection was conducted daily immediately after optic nerve injury.

Primary culture and replating culture of DRG neurons were performed as following protocols. L3-L5 dorsal root ganglia (DRGs) were dissected from adult WT mice and digested in 0.5% collagenase for 90 minutes. Subsequently, the digestion medium was replaced with culture medium (Neurobasal-A supplemented with penicillin-streptomycin and B-27). The digested DRGs were then triturated with a pipette 20-30 times to isolate DRG neurons. The isolated DRG neurons were plated onto a 6-well plate pre-coated with Poly-D-Lysine and laminin. Primary cultured DRG neurons received 1 μM edaravone, 10 μM edaravone or DMSO vehicle treatment for one day. After then, neurons were resuspended and replated to another dish under continue treatment of vehicle/edaravone. The replating culture was terminated with 4% PFA after 20 hours. Tuj1 staining was performed to visualize DRG cell bodies and neurites.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Nrf2 Overexpression Promotes Optic Nerve Regeneration

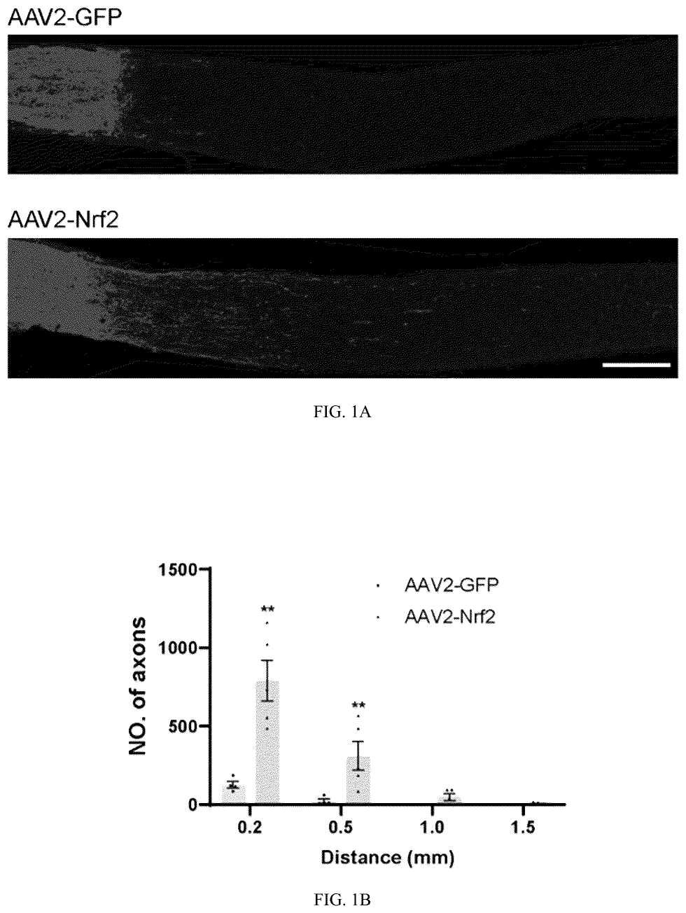

The adult wildtype mice received adeno-associated virus (AAV) carrying GFP (AAV2-hSyn-GFP) or Nrf2 (AAV2-hSyn-Nrf2) intravitreal injection to overexpression GFP in the control group and NRF2 in the experiment group. Four weeks after virus injection, optic nerve crush was conducted. The axon regeneration effect was analyzed at two weeks post injury. Unlike control group which showed negligible axon regeneration, Nrf2 overexpression significant promoted axon regeneration (FIG. 1A and FIG. 1B).

### Example 2—Nrf2 Overexpression Enhances RGC Survival

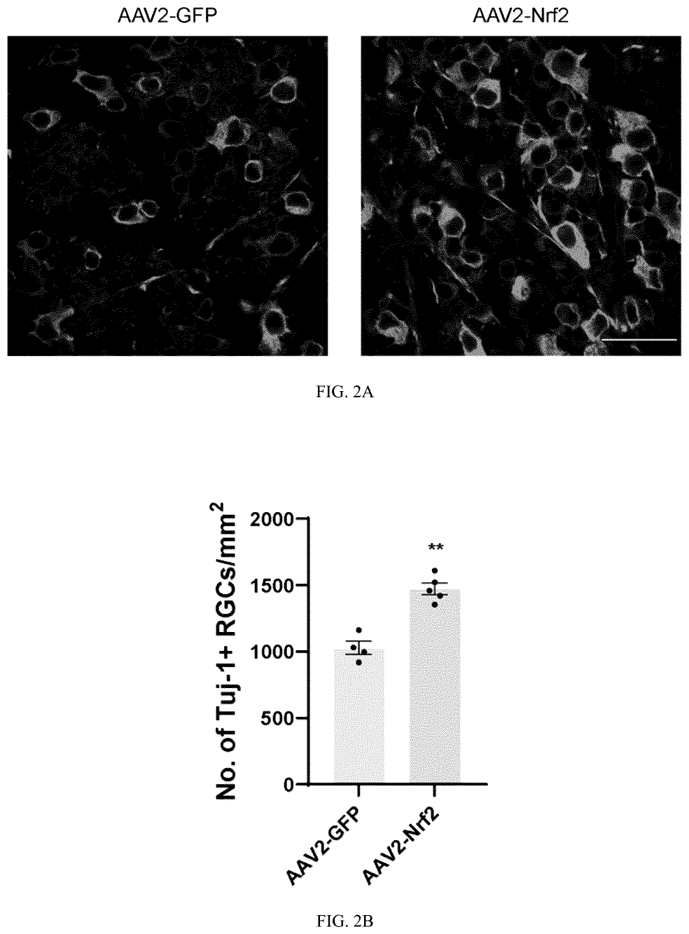

Neuronal preservation from neurotrauma or disease is essential for functional recovery. To investigate the neuronal protective effect of Nrf2, we also collected retinas at two weeks post injury. Whole-mount Tuj1 staining showed that Nrf2 overexpression significant enhanced RGCs survival (FIG. 2A and FIG. 2B).

### Example 3—Edaravone Showed Promoting Effect in DRG Replating

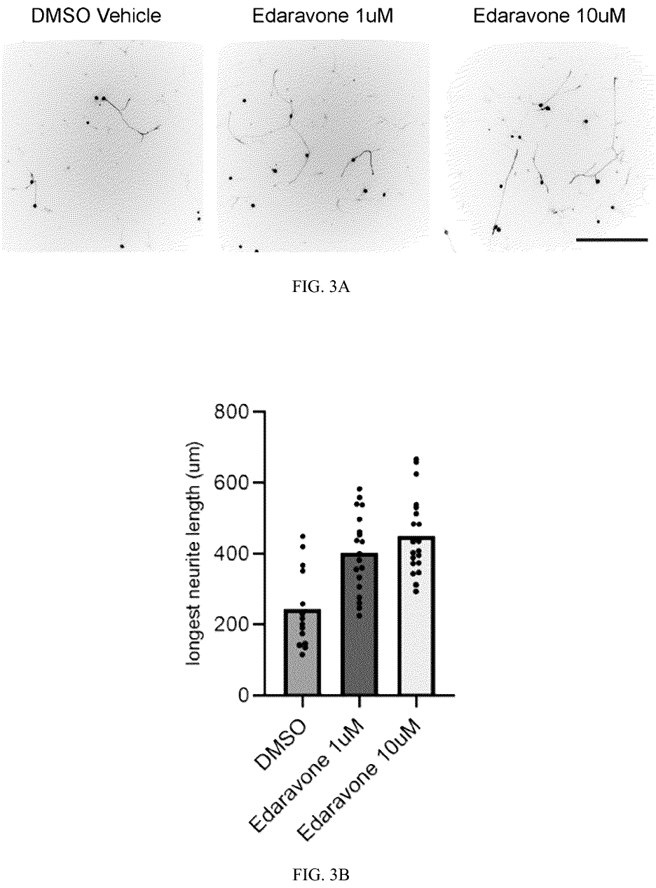

Adult mice dorsal root ganglion (DRG) neurons replating culture was used to investigate the effect of edaravone on axon growth. Primary cultured DRG neurons received 1 μM edaravone, 10 μM edaravone or DMSO vehicle treatment for one day. After then, neurons were resuspended and replated to another dish under continue treatment of vehicle/edaravone. Replated culture lasts for 20 hours for neurite growth. Compare to vehicle control group, both 1 μM edaravone and 10 μM edaravone treatment showed significant growth-promoting effect (FIG. 3A and FIG. 3B).

### Example 4—Edaravone Daily Treatment Increases RGC Survival after Optic Nerve Injury

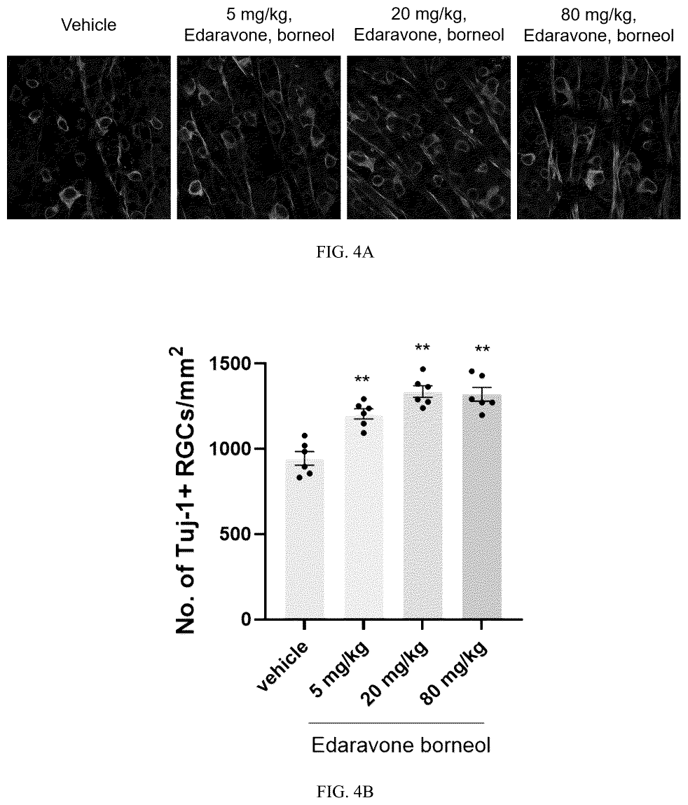

To evaluate the neuroprotective effects of edaravone, an optic nerve crush model was used. Adult mice were subjected to optic nerve crush and administered vehicle or varying dosages of edaravone via IP injection. The drugs were administered daily for two weeks before collecting the retina. Borneol was also administrated to enhance the blood-brain barrier permeability. Whole-mount Tuj1 staining revealed that daily injection of edaravone/borneol increased RGC survival following optic nerve injury. (FIG. 4A and FIG. 4B).

### Exemplary Embodiments

Embodiment 1. A method for promoting axon regeneration in a subject, the method comprising inducing Nrf2 activation.

Embodiment 2. The method of claim 1, further comprising administering to the subject a therapeutically effective amount of edaravone, whereby Nrf2 activation is induced.

Embodiment 3. The method of claim 2, wherein the subject is a mammal.

Embodiment 4. The method of claim 3, wherein the subject is a human.

Embodiment 5. The method of claim 2, wherein edaravone enhances neuron survival and promotes axon growth in the subject.

Embodiment 6. The method of claim 2, wherein edaravone is administered locally by intravitreal, intracranial, intradiscal, or systemically, by intravenous or intraperitoneal injection.

Embodiment 7. The method of claim 1, wherein the subject suffers from spinal cord injury, traumatic brain injury, optic neuropathy, stroke, or glaucoma.

Embodiment 8. The method of claim 1, wherein Nrf2 activation is induced by AAV-mediated Nrf2 overexpression or Nrf2 activator.

Embodiment 9. The method of claim 2, wherein the administration of edaravone to the subject upregulates Nrf2, and wherein the upregulation of Nrf2 reduces the production of reactive-oxygen species (ROS).

Embodiment 10. The method of claim 1, wherein Nrf2 binds to antioxidant response element (AR) sites.

Embodiment 11. The method of claim 2, wherein the edaravone is administered at a dose of about 0.1 mg/kg to about 100 mg/kg.

Embodiment 12. A method of treating a subject with central nervous system (CNS) injury by Nrf2 activation, comprising administering to a subject in need thereof a therapeutically effective amount of edaravone, wherein Nrf2 activation is induced by the edaravone.

Embodiment 13. The method of claim 12, wherein the subject is a mammal.

Embodiment 14. The method of claim 12, wherein the subject is a human.

Embodiment 15. The method of claim 12, wherein the administration of edaravone enhances neuron survival and promotes axon growth in the subject with CNS injury.

Embodiment 16. The method of claim 12, wherein edaravone is administered locally by intravitreal, intracranial, intradiscal, or systemically, by intravenous or intraperitoneal injection.

Embodiment 17. The method of claim 12, wherein the administration of edaravone to the subject upregulates Nrf2, and wherein the upregulation of Nrf2 reduces the production of reactive-oxygen species (ROS).

Embodiment 18. The method of claim 12, wherein Nrf2 binds to antioxidant response element (AR) sites.

Embodiment 19. The method of claim 12, wherein the edaravone is administered at a dose of about 0.1 mg/kg to about 100 mg/kg.

Embodiment 20. The method of claim 12, wherein edaravone is administered in a composition at a concentration of about 5 mg/mL to about 80 mg/mL.

### REFERENCES

[1] Silver J, Miller JH. Regeneration beyond the glial scar. Nat Rev Neurosci. 5(2):146-56 (2004).
[2] E. J. Bradbury and E. R. Burnside. Moving beyond the glial scar for spinal cord repair. Nat. Commun., vol. 10, no. 1, Art. no. 1 (2019).
[3] Mukhopadhyay et al. A novel role for myelin-associated glycoprotein as an inhibitor of axonal regeneration. Neuron. 13(3):757-67 (1994).
[4] Schnell L, Schwab ME. Axonal regeneration in the rat spinal cord produced by an antibody against myelin-associated neurite growth inhibitors. Nature. 18; 343(6255):269-72 (1990).
[5] He, Z. & Jin, Y. Intrinsic control of axon regeneration. Neuron 90, 437-451 (2016).
[6] Lee et al. Sustained delivery of thermostabilized chABC enhances axonal sprouting and functional recovery after spinal cord injury. Proc Natl Acad Sci USA. 23; 107(8):3340-5 (2010).
[7] J. K. Lee et al. Assessing Spinal Axon Regeneration and Sprouting in Nogo-, MAG-, and OMgp-Deficient Mice. Neuron, vol. 66, no. 5, pp. 663-670 (2010).
[8] J. K. Lee et al. Reassessment of Corticospinal Tract Regeneration in Nogo-Deficient Mice. J. Neurosci., vol. 29, no. 27, pp. 8649-8654 (2009).
[9] Zheng et al. Lack of Enhanced Spinal Regeneration in Nogo-Deficient Mice. Neuron, vol. 38, no. 2, pp. 213-224 (2003).
[10] Park et al. Promoting axon regeneration in the adult CNS by modulation of the PTEN/mTOR pathway. Science, vol. 322, no. 5903, pp. 963-966 (2008).
[11] P. D. Smith et al. SOCS3 deletion promotes optic nerve regeneration in vivo. Neuron, vol. 64, no. 5, pp. 617-623 (2009).
[12] K. J. O'Donovan et al. B-RAF kinase drives developmental axon growth and promotes axon regeneration in the injured mature CNS. J. Exp. Med., vol. 211, no. 5, pp. 801-814 (2014).
[13] S. Belin et al. Injury-induced decline of intrinsic regenerative ability revealed by quantitative proteomics. Neuron, vol. 86, no. 4, pp. 1000-1014 (2015).
[14] Wang et al. Lin28 Signaling Supports Mammalian PNS and CNS Axon Regeneration. Cell Rep., vol. 24, no. 10, pp. 2540-2552.e6 (2018).
[15] J. D. Buxbaum et al. Mutation Screening of the PTEN Gene in Patients With Autism Spectrum Disorders and Macrocephaly. Am. J. Med. Genet., vol. 144B, no. 4, pp. 484-491 (2007).
[16] M. Milella et al. PTEN: Multiple Functions in Human Malignant Tumors. Front. Oncol., vol. 5(2015).
[17] M. Gabay et al. MYC Activation Is a Hallmark of Cancer Initiation and Maintenance. Cold Spring Harb. Perspect. Med., vol. 4, no. 6 (2014).
[18] J. Balzeau et al. The LIN28/let-7 Pathway in Cancer,” Front. Genet., vol. 8, March 2017, doi: 10.3389/fgene.2017.00031.
[19] Kaufman RJ. Orchestrating the unfolded protein response in health and disease. J Clin Invest 110:1389-1398 (2002).
[20] Zhao et al. Mitochondrial dysfunction in neural injury. Frontiers in Neuroscience, vol. 13 (2019).
[21] Ryan et al. Oxidative stress in the brain and retina after traumatic injury. Front. Neurosci., Volume 17 (2023).
[22] D. A. Drechsel and M. Patel. Role of reactive oxygen species in the neurotoxicity of environmental agents implicated in Parkinson's disease. Free Radical Biology & Medicine, vol. 44, no. 11, pp. 1873-1886 (2008).
[23] Ray, P. D., Huang, B. W. & Tsuji, Y. Reactive oxygen species (ROS) homeostasis and redox regulation in cellular signaling. Cell Signal. 24, 981-990 (2012).
[24] Oh, Y. S. & Jun, H.-S. Effects of Glucagon-Like Peptide-1 on Oxidative Stress and Nrf2 Signaling. Int. J. Mol. Sci. 19, 26 (2018).
[25] US FDA: US Label Edaravone, May 2017.
[26] Hassanein et al. Edaravone and Acetovanillone Upregulate Nrf2 and PI3K/Akt/mTOR Signaling and Prevent Cyclophosphamide Cardiotoxicity in Rats. Drug Des. Dev. Ther. 14, 5275-5288 (2020).
[27] Dang et al. Edaravone ameliorates depressive and anxiety-like behaviors via Sirt1/Nrf2/HO-1/Gpx4 pathway. Journal of Neuroinflammation, vol. 19, no. 1, p. 41 (2022).
[28] A. B. Nair et al. A Simple Practical Guide for Dose Conversion Between Animal and Human,” J. Basic Clin. Pharma., vol. 87, pp. 27-31 (2016).

We claim:

1. A method for promoting axon regeneration in a subject, the method comprising inducing Nrf2 activation.

2. The method of claim 1, further comprising administering to the subject a therapeutically effective amount of edaravone, whereby Nrf2 activation is induced.

3. The method of claim 2, wherein the subject is a mammal.

4. The method of claim 2, wherein the subject is a human.

5. The method of claim 2, wherein edaravone enhances neuron survival and promotes axon growth in the subject.

6. The method of claim 2, wherein edaravone is administered locally by intravitreal, intracranial, intradiscal, or systemically, by intravenous or intraperitoneal injection.

7. The method of claim 1, wherein the subject suffers from spinal cord injury, traumatic brain injury, optic neuropathy, stroke, or glaucoma.

8. The method of claim 1, wherein Nrf2 activation is induced by AAV-mediated Nrf2 overexpression or Nrf2 activator.

9. The method of claim 2, wherein the administration of edaravone to the subject upregulates Nrf2, and wherein the upregulation of Nrf2 reduces the production of reactive-oxygen species (ROS).

10. The method of claim 1, wherein Nrf2 binds to antioxidant response element (AR) sites.

11. The method of claim 2, wherein the edaravone is administered at a dose of about 0.1 mg/kg to about 100 mg/kg.

12. A method of treating a subject with central nervous system (CNS) injury by Nrf2 activation, comprising administering to a subject in need thereof a therapeutically effective amount of edaravone, wherein Nrf2 activation is induced by the edaravone.

13. The method of claim 12, wherein the subject is a mammal.

14. The method of claim 12, wherein the subject is a human.

15. The method of claim 12, wherein the administration of edaravone enhances neuron survival and promotes axon growth in the subject with CNS injury.

16. The method of claim 12, wherein edaravone is administered locally by intravitreal, intracranial, intradiscal, or systemically, by intravenous or intraperitoneal injection.

17. The method of claim 12, wherein the administration of edaravone to the subject upregulates Nrf2, and wherein the upregulation of Nrf2 reduces the production of reactive-oxygen species (ROS).

18. The method of claim 12, wherein Nrf2 binds to antioxidant response element (AR) sites.

19. The method of claim 12, wherein the edaravone is administered at a dose of about 0.1 mg/kg to about 100 mg/kg.

20. The method of claim 12, wherein edaravone is administered in a composition at a concentration of about 5 mg/mL to about 80 mg/mL.
